(19) United States
(12) Patent Application Publication
(10) Pub. No.: US 2025/0295568 A1
(43) Pub. Date: Sep. 25, 2025

(54) ORAL CARE COMPOSITION

(71) Applicant: EVOTOOTH PLATINUM SDN. BHD., Wilayah Persekutuan Kuala Lumpur (MY)

(72) Inventor: Beng Leong ONG, Wilayah Persekutuan Kuala Lumpur (MY)

(21) Appl. No.: 18/723,601

(22) PCT Filed: Feb. 26, 2024
(86) PCT No.: PCT/MY2024/050012

§ 371 Date: Jun. 24, 2024

(30) Foreign Application Priority Data
Feb. 8, 2024 (MY) PI2024000888

(51) Int. Cl. A61K 8/42; A61Q 11/00

(52) U.S. Cl. CPC A61K 8/42; A61Q 11/00; A61K 2800/30; A61K 2800/92

(57) ABSTRACT

A non-abrasive oral care composition includes a fragrance for imparting aroma and an additive for imparting additional properties. The oral care composition forms a protective coating on an applied dental structure, the oral care composition further comprises a type of plant-derived anionic surfactant and a type of plant-derived non-ionic surfactant, wherein the type of plant-derived anionic surfactant is sodium cocoyl apple amino acid. Optionally, the oral care composition further comprises a type of plant-derived amphoteric surfactant for stabilizing the composition and enhancing coating and foaming. The composition further includes an essential oil for imparting flavour, a menthol for providing a cooling sensation, and a sweetener for imparting pleasant taste to the composition, and a preservative for preventing the growth of microorganisms.

### FIELD OF INVENTION

The present invention generally relates to a non-abrasive oral care composition. In particular, the present invention describes a non-abrasive plant-derived environment sustainable oral care composition that can form a protective coating on the dental structure in which it is applied.

### BACKGROUND OF THE INVENTION

Conventional toothpaste has a lot of flaws that are inconvenient to humans. One of the shortcomings is abrasiveness. Majority of the toothpaste available in the industry contains hydrated silica or microbead, thus prolonged brushing with this kind of toothpaste will result in abrasion or thinning on the tooth surface, as shown in FIGS. 1a, 1b and 1c. Tooth abrasion refers to a progressive and physical tooth substance loss that begins in the enamel and then progresses to dentine and pulp. Abrasion will cause exposure of dental pulp which is the innermost tooth layer that contains blood vessels and nerves; thus, penetration of pulp will cause the teeth to be non-vital. If the abrasion cavity is untreated, it will cause the tooth to become carious. Furthermore, the hydrated silica microbead is harder than enamel and dentine, hence brushing with the toothpaste will cause scratches and increase the surface roughness on tooth surface.

Other than that, most of the toothpaste in the industry contains sodium lauryl ether sulphate (SLES) or sodium lauryl sulphate (SLS) which may lead to harsh conditions in the oral cavity. SLES is derived from sulphuric acid and is harsh on the fragile lip mucosa by extracting the protective natural oil from the mucosa surface. This will lead to an undesirable condition which is dry lip symptom as shown in FIGS. 1d, 1e and 1f. SLES and Poloxamer 407 found in the conventional toothpaste also requires longer time to biodegrade and can be harmful to the marine ecosystem. Other than that, the chemical surfactants in conventional toothpaste pose a risk of interfering with the normal oral flora.

Next, most toothpaste contains fluoride which may lead to enamel fluorosis, skeletal fluorosis and neurological disorder. Moreover, there is no warning printed on toothpaste indicating that fluoride ingestion can cause teeth enamel fluorosis and invisible skeletal fluorosis. Fluorosis enamel will weaken the tooth structure and increase its porosity, rendering the teeth susceptible to straining and abrasion cavities.

Last but not least, conventional toothpaste will lead to environmental degradation in ways such as: i) microplastic pollution. Microplastics or microbeads which are not biodegradable are largely used in toothpaste; and ii) chemical pollution. An antibacterial agent found in toothpaste, triclosan, accumulates in water bodies, thus negatively affecting the aquatic ecosystems.

Thus, there is a need for a new product that can overcome these shortcomings while still providing the essential features of toothpaste. Hence, the present invention proposed an oral care composition where its inventive features can effectively prevent the flaws of conventional toothpaste.

A prior art document WO2023044471A1 discloses a jammed oil-in-water emulsion toothpaste composition. The jammed oil-in-water emulsion in this prior art has foaming properties. Besides that, it also uses natural surfactants, including anionic surfactant, non-ionic surfactant and amphoteric surfactant as well as combinations thereof. Besides that, the toothpaste composition can be free of, essentially free of, and/or substantially free of abrasive. The jammed oil-in-water composition can provide abrasive-free cleaning as a) it can quickly release cleaning or bleaching agents that may be present in the aqueous phase, and b) it can have a majority hydrophobic phase that can remove or bleach stains, plaque, tartar, biofilm, and/or bacteria through an oil pulling or bleaching mechanism. Other than that, the jammed oil-in-water toothpaste composition in this prior art may be free of, essentially free of, and/or substantially free of sulfate, alkyl sulfate, and/or sodium lauryl sulfate, as some consumers have a perception that these surfactants may lead to harsh conditions in the oral cavity, thus preventing dry lip symptoms. However, the jammed oil-in-water toothpaste composition in this prior art does not form a protective coating on the enamel, cervical dentine, and ceramic or zirconia crown surfaces. It also uses poloxamer as a non-ionic surfactant and thickening agent which requires longer time to biodegrade. It also contains fluoride which may cause enamel fluorosis, skeletal fluorosis and neurological disorder.

A Korean prior art document KR20180071348A discloses a mouthwash comprising partially hydrolyzed plant protein, which, for example, restores or prevents tooth erosion, promotes tooth remineralization, and/or enhances the anti-caries effect of fluoride. This invention will produce foam. However, it does not form a protective coating on the enamel, cervical dentine, and ceramic or zirconia crown surfaces. It also uses abrasives such as hydrated silica as a polishing agent. The anionic surfactants used in this invention include sodium lauryl sulphate and sodium ether lauryl sulphate which are harsh on fragile lip mucosa. Besides that, it also uses poloxamers such as poloxamer 407, poloxamer 188 and poloxamer 338 as non-ionic surfactants. It also contains fluoride.

Neither of the prior arts above forms a protective coating on the surface in which the inventions are applied, particularly the parts in the oral cavity such as enamel, cervical dentine, and ceramic or zirconia crown surfaces. Protective coating is advantageous as it can increase the surface layer thickness, prevent abrasion, protect against staining, protect against acidic diet as well as reduce cervical dentine hypersensitivity. The present invention can effectively provide this feature; thus, it is an innovative invention in the oral care sector.

### SUMMARY OF THE PRESENT INVENTION

The present invention features a non-abrasive oral care composition, comprising a fragrance for imparting aroma and a natural additive for imparting additional properties, characterized in that the oral care composition forms a protective coating on an applied dental structure, the oral care composition further comprises a type of plant-derived anionic surfactant and a type of plant-derived non-ionic surfactant, wherein the type of plant-derived anionic surfactant is sodium cocoyl apple amino acid.

Preferably, the oral care composition further comprises a type of plant-derived amphoteric surfactant for stabilizing the composition and enhancing coating and foaming.

Preferably, the type of plant-derived amphoteric surfactant is cocamidopropyl betaine.

Preferably, the composition range of the plant-derived amphoteric surfactant is 0.1 to 3 wt %.

Preferably, the composition range of the plant-derived anionic surfactant is 0.3 to 6 wt %.

Preferably, the type of plant-derived non-ionic surfactant is lauryl glucoside or vegetable glycerin.

Preferably, the composition range of the plant-derived non-ionic surfactant is 0.1 to 3 wt %.

Preferably, the composition is a liquid that dispenses into a foam.

Preferably, the composition further comprises an essential oil for imparting flavour, a menthol for providing a cooling sensation, and a sweetener for imparting pleasant taste to the composition.

Preferably, the composition comprises a preservative for preventing the growth of microorganisms.

The present invention consists of features and a combination of parts from now on fully described and illustrated in the accompanying drawings; it being understood that various changes in the details may be made without departing from the scope of the invention or sacrificing any of the advantages of the present invention.

### BRIEF DESCRIPTION OF THE DRAWINGS

To further clarify various aspects of some embodiments of the present invention, a more particular description of the invention will be rendered by referencing specific embodiments, which are illustrated in the appended drawings. It is appreciated that these drawings depict only typical embodiments of the invention and are therefore not to be considered limiting of its scope. The invention will be described and explained with additional specificity and detail through the accompanying drawings in which:

FIGS. 1a-1c illustrate clinical photos of abrasion on tooth surface after using toothpaste.

FIGS. 1d-1f illustrate clinical photos of dry lip symptoms caused by SLES and SLS in toothpaste.

FIG. 2a illustrates in vitro Scanning Electron Microscope (SEM) images under 100×, 500× and 1000× magnification of the difference in smoothness of the enamel rods on a tooth's surface before brushing or baseline, brushing with toothpaste and brushing with invention tooth foam in a preferable composition of 3 wt % of plant-derived anionic surfactant, 0.8 wt % of plant-derived non-ionic surfactant as well as 0.7 wt % of plant-derived amphoteric surfactant, where a noticeable coating is shown on the tooth's surface when brushing with invention tooth foam.

FIG. 2b illustrates in vitro Scanning Electron Microscope (SEM) images under 100×, 500× and 1000× magnification which shows lesser scratches on enamel surface after applying tooth foam in a preferable composition of 3 wt % of plant-derived anionic surfactant, 0.8 wt % of plant-derived non-ionic surfactant as well as 0.7 wt % of plant-derived amphoteric surfactant.

FIG. 2c illustrates in vitro Scanning Electron Microscope (SEM) images under 100×, 500× and 1000× magnification of tooth enamel surface before brushing or baseline, after brushing with toothpaste and after brushing with invention tooth foam in a preferable composition of 3 wt % of plant-derived anionic surfactant, 0.8 wt % of plant-derived non-ionic surfactant as well as 0.7 wt % of plant-derived amphoteric surfactant, where scratches are significantly reduced after brushing with invention tooth foam as a protective coating filled into the micro scratches and enamel rod.

FIGS. 3a-3d illustrate images of an in vivo Optical Coherence Tomography (OCT) test performed on enamel which show that the thickness of the tooth foam coating increased with each successive application of the tooth foam in a preferable composition of 3 wt % of plant-derived anionic surfactant, 0.8 wt % of plant-derived non-ionic surfactant as well as 0.7 wt % of plant-derived amphoteric surfactant.

FIGS. 3e-3f illustrate images of an in vivo Optical Coherence Tomography (OCT) test performed on enamel which show that initially plaque was present at the baseline and following the application of tooth foam in a preferable composition of 3 wt % of plant-derived anionic surfactant, 0.8 wt % of plant-derived non-ionic surfactant as well as 0.7 wt % of plant-derived amphoteric surfactant, a thick coating of tooth foam was observed on the tooth surfaces.

FIGS. 3g-3i illustrate images of an in vivo Optical Coherence Tomography (OCT) test performed on ceramic crown which show that initially plaque was present at the baseline and following the application of tooth foam in a preferable composition of 3 wt % of plant-derived anionic surfactant, 0.8 wt % of plant-derived non-ionic surfactant as well as 0.7 wt % of plant-derived amphoteric surfactant, a thick coating of tooth foam was observed on the tooth surfaces which further increase in thickness after brushing with toothpaste and applying tooth foam.

FIGS. 4a-4d illustrate in vivo Optical Coherence Tomography (OCT) test images of dentine surface of cervical abrasion baseline, after brushing with invention tooth foam in a preferable composition of 3 wt % of plant-derived anionic surfactant, 0.8 wt % of plant-derived non-ionic surfactant as well as 0.7 wt % of plant-derived amphoteric surfactant, after application of citric acid for 30 seconds and after application of citric acid for 60 seconds.

FIG. 4e illustrates in vivo Optical Coherence Tomography (OCT) test images of reapplication of invention tooth foam in a preferable composition of 3 wt % of plant-derived anionic surfactant, 0.8 wt % of plant-derived non-ionic surfactant as well as 0.7 wt % of plant-derived amphoteric surfactant on the citric acid treated dentine surface of cervical abrasions, where the surface coating thickness increases again significantly after reapplication of tooth foam.

FIGS. 5a-5e illustrate in vivo Optical Coherence Tomography (OCT) images showing the presence of tooth foam in a preferable composition of 3 wt % of plant-derived anionic surfactant, 0.8 wt % of plant-derived non-ionic surfactant as well as 0.7 wt % of plant-derived amphoteric surfactant on enamel, ceramic crown and dentine even after certain time intervals, indicating the durability of the tooth foam.

### DETAILED DESCRIPTION OF THE INVENTION

The present invention generally relates to a non-abrasive oral care composition. In particular, the present invention describes a non-abrasive plant-derived environment sustainable oral care composition that can form a protective coating on the dental structure in which it is applied.

From now on, the non-abrasive oral care composition according to the present invention will be described in detail according to the preferred embodiments. It is to be understood that limiting the description to the preferred embodiments of the invention is merely to facilitate discussion of the present invention, and it is envisioned without departing from the scope of the appended claims.

In the preferred embodiment of the present invention, the non-abrasive oral care composition is preferably a liquid that dispenses into a foam. The foam form is particularly beneficial when the composition is used in oral care. This is because tooth brushing is normally divided into four quadrants, which are upper left and right, and lower left and right. When toothpaste is used, it will be depleted when brushing the last quadrant which will affect the cleaning efficiency. On the other hand, the foam of the present invention comes in tooth foam bottles which require two full pumps for the age of twelve and above and one full pump for those below the age of twelve. The recommended way is to pump foam into the oral cavity, then gargle the foam for five to ten seconds to spread the foam around all four quadrants simultaneously. This prevents the early depletion of toothpaste before brushing all four quadrants. The present invention uses refillable packaging and encourages reusable foam bottle, thus it is environmentally friendly.

In the preferred embodiment of the present invention, the non-abrasive oral care composition comprises a fragrance for imparting aroma. Examples of the fragrance is green tea oil, vanilla essence, butter milk, coffee essence, fruit, tea, lime, anise, chamomile, litsea, cucumber mint, grapefruit and tangerine. Preferably, the fragrance is obtained from a natural resource. This is because natural fragrance does not contain chemicals or toxins which are harmful when ingested or in contact with the skin. Other than that, the non-abrasive oral care composition also comprises a natural additive for imparting additional properties. Examples of the natural additive are sodium chloride (to balance the sweetness), white vinegar (as a foam finer), allantoin (anti-irritation purpose), carrageenan, guar gum, xanthan gum, agar, ascorbic acid, gelatin, carnauba wax and lecithin. Next, the non-abrasive oral care composition further comprises an essential oil for imparting flavour. Examples of essential oil include spearmint oil, jasmine oil, chamomile oil, lavender oil, patchouli, peppermint oil, tea tree oil, eucalyptus oil, frankincense oil, orange oil, lemongrass oil and bergamot oil. Besides that, the non-abrasive oral care composition also comprises a menthol for providing a cooling sensation. Preferably, the menthol is obtained from menthol crystal which can provide a minty flavour, corn mint oil, peppermint oil or other natural resources. Preferably, the non-abrasive oral care composition also comprises a preservative for preventing the growth of microorganisms. Examples of the preservative are 99% ethanol, propyl paraben, sodium benzoate, ethylparaben and methylparaben. Besides that, the non-abrasive oral care composition also comprises a sweetener for imparting pleasant taste to the composition. Examples of sweetener is sodium saccharin, brown rice syrup, agave nectar, honey, date sugar, molasses, maple syrup, blackstrap molasses, stevia and sorghum syrup.

This non-abrasive oral care composition is characterized in that it further comprises a type of plant-derived anionic surfactant and a type of plant-derived non-ionic surfactant for forming a protective coating on an applied dental structure. Preferably, the non-abrasive oral care composition further comprises a type of plant-derived amphoteric surfactant for stabilizing the composition and enhancing coating and foaming. Preferably, the type of plant-derived amphoteric surfactant is cocamidopropyl betaine. The reason why it is chosen is because it can also function as a thickening agent besides being an excellent foaming agent. It also has a bactericidal effect. Preferably, the composition range of the plant-derived amphoteric surfactant is 0.1 to 3 wt %. More preferably, the composition range is 0.5 to 1 wt %.

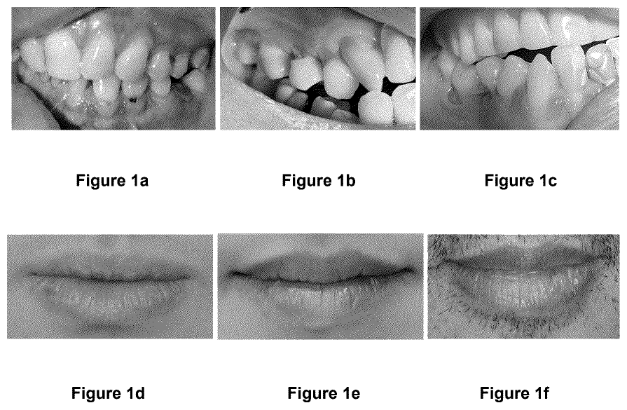

Preferably, the type of plant-derived anionic surfactant is sodium cocoyl apple amino acid. Alternatively, other types of amino acid can also be used as anionic surfactant, such as sodium methyl cocoyl taurate, potassium cocoyl glycinate, sodium taurine cocoyl methyl taurate, sodium stearoyl glutamate and sodium cocoyl glycinate. Conventional oral care compositions available in the industry like toothpaste use anionic surfactants such as sodium lauryl ether sulphate (SLES) or sodium lauryl sulphate (SLS) which is a surfactant that produces foaming. However, SLES and SLS are derived from sulphuric acid which is harsh on the fragile lip mucosa as it extracts natural oil from the mucosa surface. This results in dry lip symptoms as shown in FIGS. 1d to 1f which will bring undesirable consequences such as cracked lips, bad breath, bleeding, mouth sores, taste disorders, thick saliva, increased thirst, trouble chewing or swallowing, gum disease, redness, swelling, and problems wearing dentures.

The present invention uses sodium cocoyl apple amino acid as the anionic surfactant which is gentle to the oral and lip mucosa. It is also hydrating and can improve the surface texture that it is applied to. As sodium cocoyl apple amino acid is a plant-derived natural surfactant, it is biodegradable, thus it can contribute to environmental sustainability. When used in oral care, natural plant-derived surfactant will not interrupt the balance of oral flora. Preferably, the composition range of the plant-derived anionic surfactant is 0.3 to 6 wt %. More preferably, the composition range is 2 to 3 wt %.

Preferably, the type of plant-derived non-ionic surfactant is lauryl glucoside. The reason why lauryl glucoside is chosen as the non-ionic surfactant is due to it being an excellent co-surfactant with good foaming capacity and is gentle to mucosal. Another alternative that can be used as non-ionic surfactant is vegetable glycerin. Apart from acting as a non-ionic surfactant, vegetable glycerin can also act as an emulsifier as it has excellent emulsifying properties. Alternatively, other members of the alkyl polyglucosides (APG) functional group can also be used as non-ionic surfactant. Some examples include coco glucoside, caprylyl-capryl glucoside, caprylyl-decyl glucoside, hexyl glucoside, isooctyl glucoside and decyl glucoside. Preferably, the composition range of the plant-derived non-ionic surfactant is 0.1 to 3 wt %. More preferably, the composition range is 0.5 to 1 wt %.

The inventive feature of the non-abrasive oral care composition is it forms a protective coating on an applied dental structure. This protective coating is a notable innovative feature as it provides several solutions to existing problems incurred by current oral care products, especially toothpaste. One of the solutions that the protective coating provides is it can increase surface smoothness and prevent abrasion of the tooth. This is because the protective coating forms on enamel, cervical dentine, and ceramic or zirconia crown surfaces, where continuous brushing with the foam twice daily can produce the long-lasting protective coating which fills up the micropores of enamel rods and dentinal tubules. This increases the surface smoothness of the tooth and prevents abrasion.

Conventional toothpaste contains hydrated silica or microbead with hardness value of 5-7 on Mohs scale. Enamel hardness is 5 on Mohs scale whereas dentine is 3-4 on Mohs scale. Hence, prolonged brushing with toothpaste can cause thinning or abrasion on the tooth surface, especially exposed dentine at the cervical root surface. There are many adult patients suffering from multiple abrasion cavities due to brushing with toothpaste containing hydrated silica or microbeads. The abrasion causes a lot of scratches on the tooth surface. The protective coating of this oral care composition will fall into the scratches and cover them, thus solving this issue.

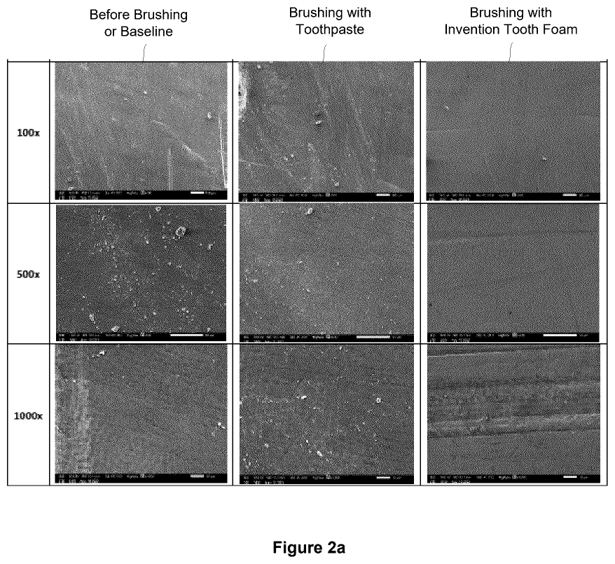

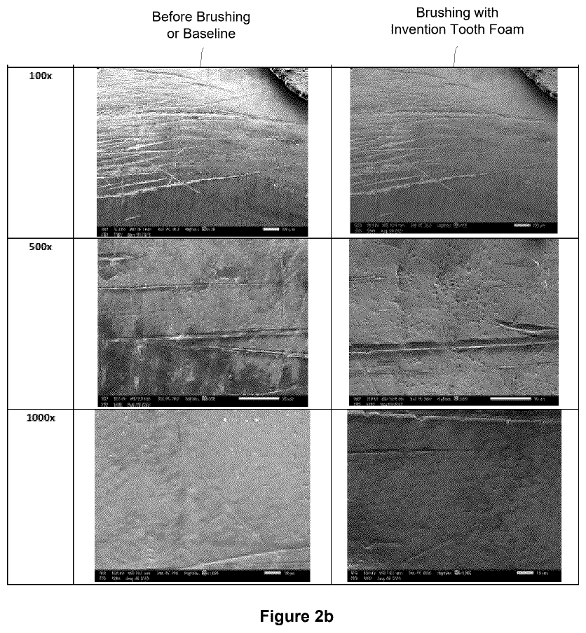

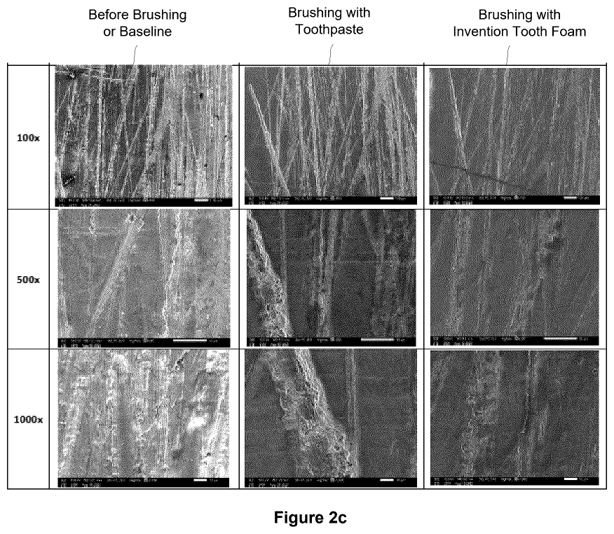

The effect of the protective coating of the oral care composition on preventing abrasion is proven in FIGS. 2a, 2b and 2c. FIG. 2a illustrates in vitro Scanning Electron Microscope (SEM) images under 100×, 500× and 1000× magnification of the difference in smoothness of the enamel rods on a tooth's surface before brushing or baseline, brushing with toothpaste and brushing with invention tooth foam, where a noticeable coating is shown on the tooth's surface when brushing with invention tooth foam. It is seen that after tooth brushing with toothpaste (middle column of FIG. 2a), the enamel rods appeared to be smoother. However, following the application of tooth foam (right column of FIG. 2a), the enamel rods exhibited an even more pronounced smoothness, along with the emergence of a noticeable coating on the tooth's surface. As for FIG. 2b, it illustrates in vitro Scanning Electron Microscope (SEM) images under 100×, 500× and 1000× magnification which shows lesser scratches on enamel surface after applying tooth foam (right column of FIG. 2b) compared to the initial abrasions before brushing (left column of FIG. 2b). Further, FIG. 2c illustrates in vitro Scanning Electron Microscope (SEM) images under 100×, 500× and 1000× magnification of tooth enamel surface before brushing or baseline (left column of FIG. 2c) which show scratches on the enamel surface and significantly lesser scratches after brushing with invention tooth foam (right column of FIG. 2c) as a protective coating filled into the micro scratches and enamel rod.

The ability of the protective coating to prevent abrasion and reduce surface roughness is further proven through an in vitro 3D Alicona test performed on extracted teeth. The results are shown in Table 1 and Table 2.

TABLE 1
Surface Roughness Data (A1, A2, B1, B2, C1, C2)

| | | | Tooth Foam to |
|---|---|---|---|
| Sample ID | Baseline | Tooth Foam | Toothpaste |
| A1 | 0.226 μm | 0.192 μm | 0.198 μm |
| A2 | 0.200 μm | 0.185 μm | 0.195 μm |
| B1 | 0.301 μm | 0.254 μm | 0.263 μm |
| B2 | 0.654 μm | 0.333 μm | 0.475 μm |
| C1 | 0.354 μm | 0.238 μm | 0.345 μm |
| C2 | 0.455 μm | 0.367 μm | 0.384 μm |
| Mean & Std | 0.365 μm ± | 0.262 μm ± | 0.310 μm ± |
| Deviation | 0.154 μm | 0.068 μm | 0.102 μm |

TABLE 2
Surface Roughness Data (D1, D2, E1, E2, F1, F2, G1, G2)

| | | | Toothpaste to |
|---|---|---|---|
| Sample ID | Baseline | Toothpaste | Tooth Foam |
| D1 | 0.225 μm | 0.285 μm | 0.272 μm |
| D2 | 0.184 μm | 0.266 μm | 0.266 μm |
| E1 | 0.226 μm | 0.264 μm | 0.237 μm |
| E2 | 0.256 μm | 0.264 μm | 0.251 μm |
| F1 | 0.307 μm | 0.342 μm | 0.321 μm |
| F2 | 0.316 μm | 0.353 μm | 0.344 μm |
| G1 | 0.197 μm | 0.262 μm | 0.240 μm |
| G2 | 0.231 μm | 0.270 μm | 0.248 μm |
| Mean & Std | 0.243 μm ± | 0.288 μm ± | 0.272 μm ± |
| Deviation | 0.045 μm | 0.035 μm | 0.037 μm |

As shown in Table 1, the surface roughness data reveals noteworthy change in tooth enamel texture. At the baseline, the mean surface roughness was measured at 0.365 μm±0.154 μm. Following the use of tooth foam, there was an obvious reduction in roughness, with a mean of 0.262 μm±0.068 μm, indicating a smoother tooth surface. Interestingly, when tooth brushing was performed after the application of toothpaste, the surface roughness slightly increased to 0.310 μm±0.102 μm, though it remained below the baseline value, suggesting that this combination may still contribute to a smoother enamel texture compared to the initial state.

As for Table 2, initially at the baseline, the mean surface roughness was measured at 0.243 μm±0.045 μm. Subsequent to the application of toothpaste, the surface roughness increased to a mean of 0.288 μm±0.035 μm, indicating a slight increase in surface texture. Interestingly, when tooth brushing was followed by tooth foam application, the surface roughness decreased to 0.272 μm±0.037 μm, suggesting that this combination contributed to a smoother enamel texture compared to the baseline, although it remained slightly elevated compared to the initial measurement after toothpaste alone. Besides that, this protective coating formed by the oral care composition is able to reduce extrinsic tooth staining caused by food, drinks, and tobacco smoking as it seals up the enamel rods and dentinal tubules.

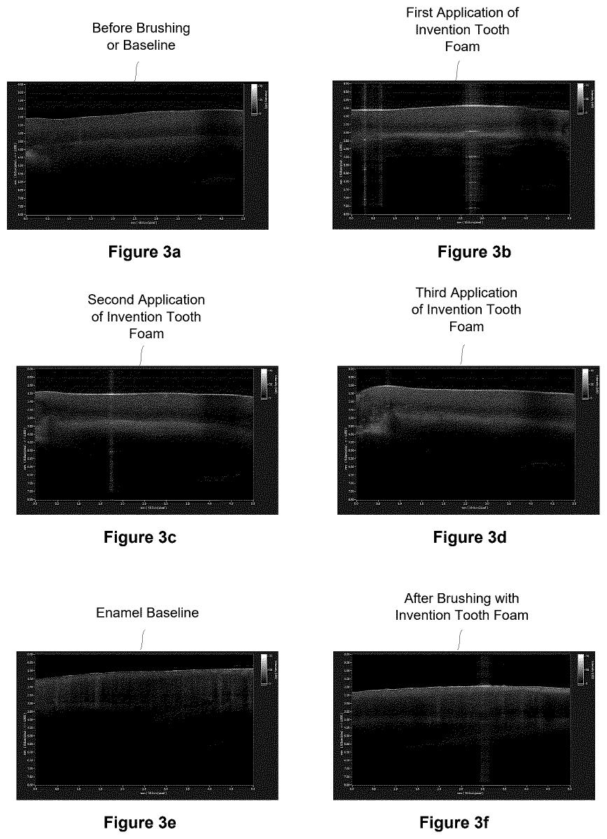

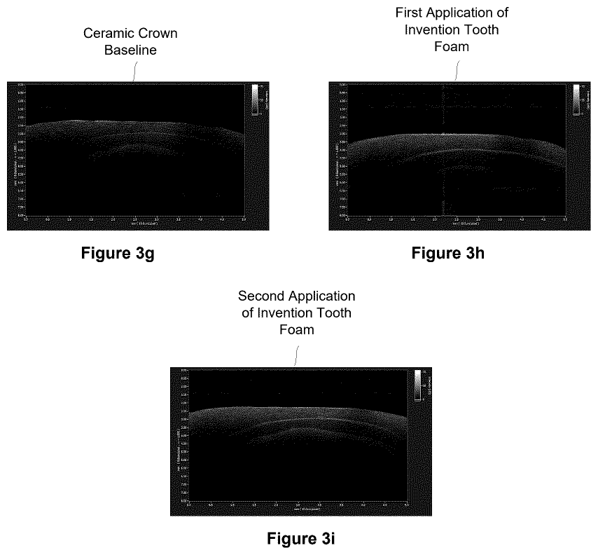

The formation of the protective coating after tooth foam is applied is shown in FIGS. 3a to 3i. FIGS. 3a-3d illustrate images of an in vivo Optical Coherence Tomography (OCT) test performed on enamel which shows that the thickness of the tooth foam coating increased with each successive application of the tooth foam, that is, from baseline plaque thickness of 32 μm to 48 μm (first application of tooth foam), 50 μm (second application of tooth foam) and 56 μm (third application of tooth foam). Next, FIGS. 3e-3f illustrate images of an in vivo OCT test performed on enamel which show that initially plaque was present at the baseline (plaque thickness 24 μm). Following the application of tooth foam, a thick coating of tooth foam was observed on the tooth surfaces. The thickness increased to 48 μm. As for FIGS. 3g-3i, images of an in vivo OCT test performed on ceramic crown illustrate that initially plaque was present at the baseline (plaque thickness 24 μm) and following the application of tooth foam, a thick coating of tooth foam was observed on the tooth surfaces (thickness increased to 40 μm) which further increase in thickness after brushing with toothpaste and applying tooth foam, that is, increased to 40.79 μm.

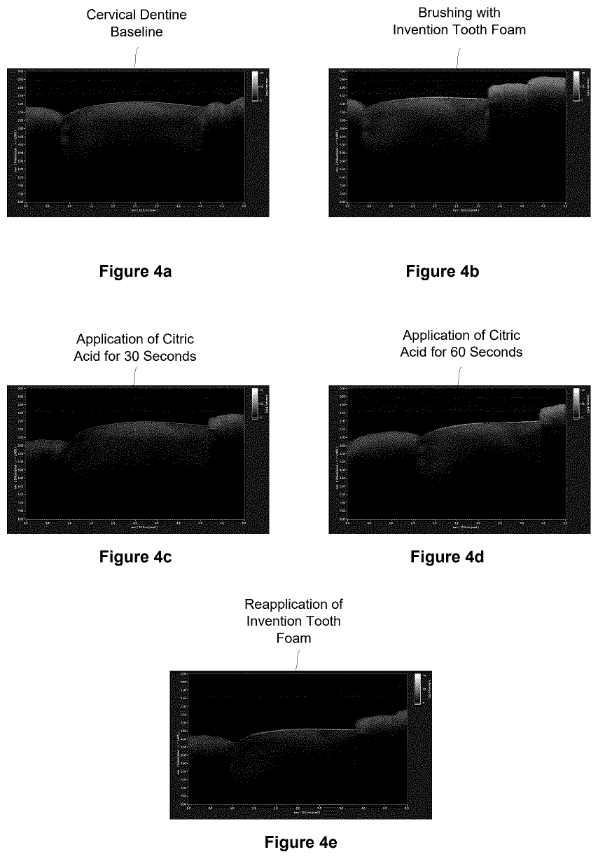

Other than that, the protective coating formed by this oral care composition is able to protect the cervical dentine from erosion caused by acidic food and drinks. This is because acid reacts stronger with inorganic substances while the oral care composition uses organic ingredients. This is proven in the in vivo Optical Coherence Tomography (OCT) test performed on the dentine surface of cervical abrasions as shown in FIGS. 4a to 4e where citric acid is added on the dentine surface of cervical abrasions of three teeth samples. Two of the teeth are upper first premolars, and one of them was an upper canine tooth. The baseline OCT images showed on the dentine surface of cervical abrasions indicated that the surface layer thickness ranged from 4 μm to 16 μm. The surface layer thickness on the dentine surface of cervical abrasions increased after the application of the tooth foam and ranged from 18 μm to 28 μm. The dentine surface of cervical abrasions was then subjected to citric acid treatment using lime juice for 30 seconds and 60 seconds. The surface layer thickness decreased after the application of lime juice to the dentine surface of cervical abrasions and ranged from 13 μm to 26 μm. This was followed by the application of the tooth foam on the citric acid-treated dentine surface of cervical abrasions which increased the surface layer thickness which ranged from 20 μm to 25 μm thickness. The study suggests that the application of tooth foam increased the surface layer thickness of the dentine surface of cervical abrasions, and citric acid treatment with lime juice decreased the thickness. However, when tooth foam was applied to the dentine surface of cervical abrasions treated with citric acid, the surface layer thickness increased again. The study implies a dynamic interaction between the tooth foam and citric acid treatment in affecting the surface layer thickness of the dentine surface of cervical abrasions. This indicates that tooth foam plays a role in safeguarding the dentine surface of the cervical abrasions.

As the protective coating formed by this oral care composition is able to protect the cervical dentine from erosion caused by acidic food and drinks, it can also reduce cervical dentine hypersensitivity as the protective coating reduces osmotic activity in dentinal tubules that trigger dentine hypersensitivity. This is much desired by people with this dental condition as dentine hypersensitivity inflicts serious pain when eating something acidic, hot or cold, thus limiting the diet options of the patients, resulting in significant deterioration of life quality.

Another plus side of the protective coating formed by the oral care composition is it is versatile. The protective coating can form on natural dental structures such as enamel, cervical dentine and ceramic or zirconia crown surfaces, synthetic dental structures such as bridges, dentures and dental implants as well as composites. Hence, it can be widely applied in various dental structures, making it a valuable product in the oral care industry.

Besides that, majority of the conventional toothpaste contains fluoride which poses a risk of ingestion, especially in children. Prolonged ingestion of fluoride can cause enamel fluorosis, skeletal fluorosis, and cognitive disorder. Fluoride is also found to be neurotoxic. The present invention does not contain fluoride, thus it is beneficial to the tooth structure, skeletal structure as well as the neurological system. The function of fluoride in conventional toothpaste which is preventing tooth decay is substituted by the protective coating formed by the present invention.

Other than that, chemical surfactants such as SLES and Poloxamer 407 found in toothpaste require longer time to biodegrade and can be harmful to the marine ecosystem. As the present invention uses natural surfactants that are biodegradable, the environment can be preserved, ensuring environmental sustainability.

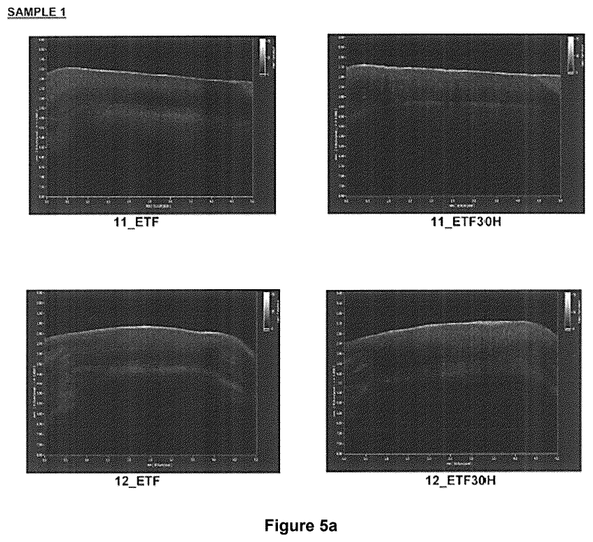

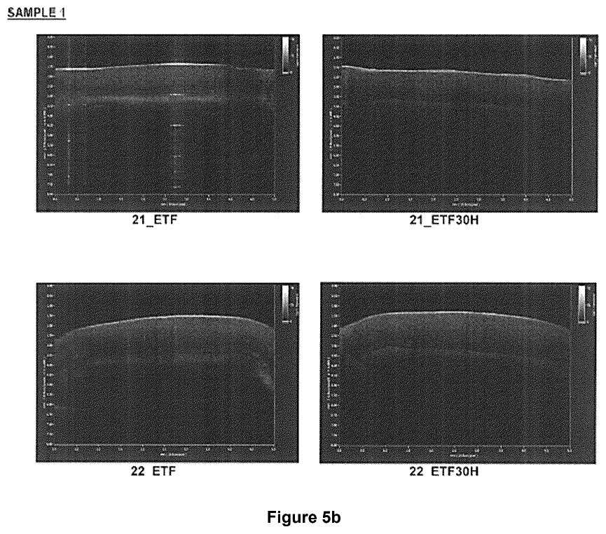

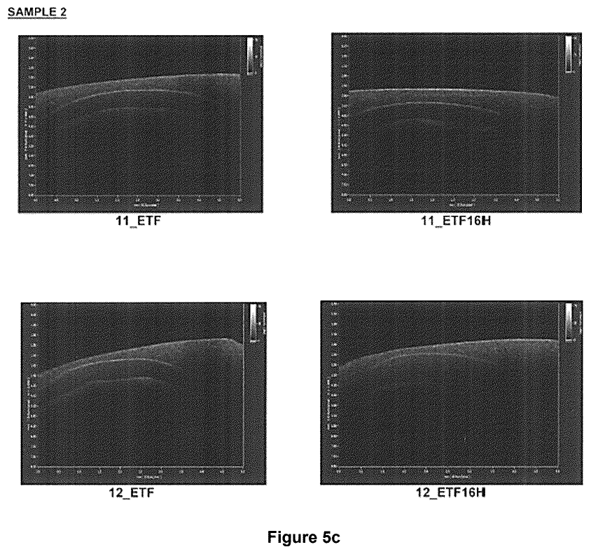

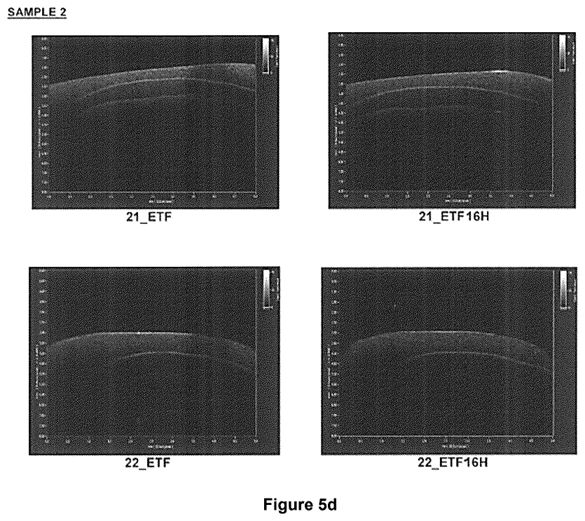

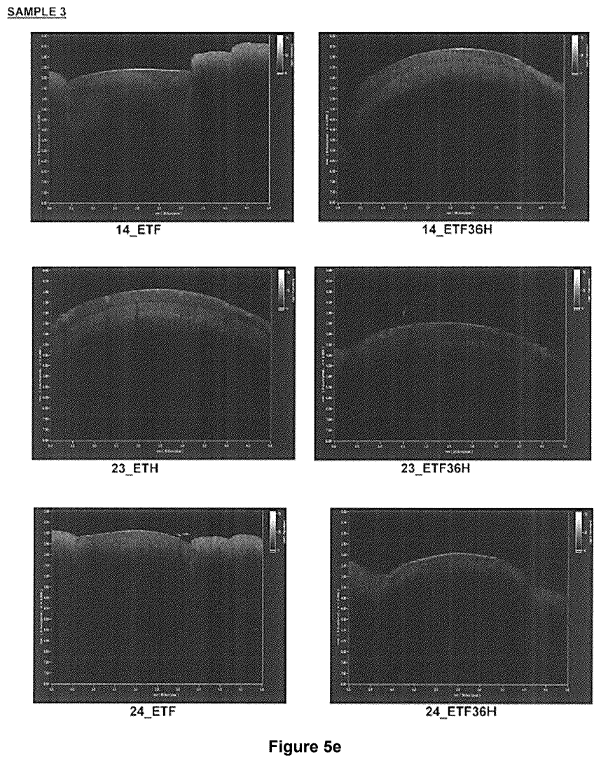

The durability of the tooth foam is also shown in FIGS. 5a to 5e, through an in vivo Optical Coherence Tomography (OCT) test. This study comprises three cases, with OCT images captured on four teeth in two cases and on three crowns in the third case. In the first case, OCT images were taken 30 hours after applying the foam on the enamel surface. In the second case, the baseline OCT images were followed by images at 16 hours post-application of the foam on ceramic crown surface. The third case involves capturing OCT images at baseline and 36 hours after using the foam on the dentine surfaces.

For the first case, OCT images were captured on teeth 11_ETF, 12_ETF, 21_ETF, and 22 ETF (FIGS. 5a and 5b), depicting the presence of tooth foam on all four enamel surfaces both at baseline and 30 hours after the application of tooth foam. The thickness of the tooth foam varies, ranging from 18.66 μm to 34.66 μm at baseline and from 16.00 μm to 66.66 μm 30 hours after the application of tooth foam. For the second case, OCT images were captured on teeth 11_ETF, 12_ETF, 21_ETF, and 22_ETF (FIGS. 5c and 5d), revealing the presence of tooth foam on all four ceramic crown surfaces at both baseline and 16 hours after the application of tooth foam. The thickness of the tooth foam varied, ranging from 5.33 μm to 21.33 μm at baseline and expanding to a range of 5.33 μm to 32.00 μm 16 hours after the application of tooth foam. For the third case, OCT images were captured on the cervical abrasion dentine 14_ETF, 23_ETH, and 24_ETF (FIG. 5e), indicating the presence of tooth foam on all three dentine surfaces at both baseline and 36 hours after the application of tooth foam. The thickness of the tooth foam varied, ranging from 8.00 μm to 24.00 μm at baseline and expanding to a range of 12.00 μm to 44.00 μm 36 hours after the application of tooth foam.

Based on the findings of this study, it can be inferred that the foam demonstrates durability across various dental surfaces. In the enamel surfaces, the tooth foam remained present 30 hours post-application, exhibiting a thickness range of 16.05 μm to 80.04 μm. Similarly, on dentine surfaces, the tooth foam persisted from baseline to 36 hours, with thickness variations. Even on ceramic crown surfaces, the tooth foam was detected at both baseline and 16 hours, showcasing a range of thickness. This suggests the potential durability and sustained presence of the tooth foam on enamel, dentine, and ceramic crown surfaces, highlighting its promising properties in dental applications.

Combination of the proper ratio of the three different types of plant-derived natural surfactants (anionic surfactant, non-ionic surfactant and amphoteric surfactant) will produce a synergistic effect that lowers the surface tension of debris and allows excellent foaming properties. The synergistic effect can also provide non-abrasive effective teeth cleansing that is gentle to the oral and lip mucosal, and has the ability to protect the teeth by forming durable protective coating. Although similar results could be achieved by using only plant-derived anionic and non-ionic surfactants, the lack of amphoteric surfactant in the oral care composition will lead to reduction of the value of teeth cleansing and protective coating.

The protective coating formed by the present invention on teeth surface is a new milestone to safeguard teeth structure from acidic diet, extrinsic staining, physical abrasion from teeth brushing and to reduce teeth hypersensitivity at exposed cervical dentine. Thus, it can reduce the material wastage from treating abrasion cavity, cervical dentine hypersensitivity, extrinsic staining polishing and dry lip treatment that is caused by SLES in toothpaste. Besides that, the usage of good biodegradability plant-derived surfactants and ingredients can protect the environment. Both of these benefits brought by the present invention can achieve environmental sustainability.

The present invention may be embodied in other specific forms without departing from its essential characteristics. The described embodiments are to be considered in all respects only as illustrative and not restrictive. Therefore, the scope of the invention is indicated by the appended claims rather than by the preceding description. All changes that come within the meaning and range of equivalency of the claims are to be embraced within their scope. Throughout this specification, unless the context requires otherwise, the word “comprise,” or variations such as “comprises” or “comprising,” will be understood to imply the inclusion of a stated element or group of elements, but not the exclusion of any other element or group of elements. Thus, in the context of this specification, the term “comprising” is used in an inclusive sense and therefore should be understood as meaning “including principally, but not necessarily solely.”

1. A non-abrasive oral care composition, comprising:
a fragrance for imparting aroma; and
an additive for imparting additional properties,
wherein the oral care composition forms a protective coating on an applied dental structure, the oral care composition further comprises a type of plant-based derived anionic surfactant and a type of plant derived non-ionic surfactant, wherein the type of plant-derived anionic surfactant is sodium cocoyl apple amino acid.

2. The non-abrasive oral care composition as claimed in claim 1, wherein the oral care composition further comprises a type of plant derived amphoteric surfactant for stabilizing the composition and enhancing coating and foaming.

3. The non-abrasive oral care composition as claimed in claim 2, wherein the type of plant derived amphoteric surfactant is cocamidopropyl betaine.

4. The non-abrasive oral care composition as claimed in claim 2, wherein the composition range of the plant derived amphoteric surfactant is 0.1 to 3 wt %.

5. (canceled)

6. The non-abrasive oral care composition as claimed in claim 1, wherein the composition range of the plant derived anionic surfactant is 0.3 to 6 wt %.

7. The non-abrasive oral care composition as claimed in claim 1, wherein the type of plant derived non-ionic surfactant is lauryl glucoside or vegetable glycerin.

8. The non-abrasive oral care composition as claimed in claim 1, wherein the composition range of the plant derived non-ionic surfactant is 0.1 to 3 wt %.

9. The non-abrasive oral care composition as claimed in claim 1, wherein the composition is a liquid that dispenses into a foam.

10. The non-abrasive oral care composition as claimed in claim 1, wherein the composition further comprises an essential oil for imparting flavour, a menthol for providing a cooling sensation, and a sweetener for imparting pleasant taste to the composition.

11. The non-abrasive oral care composition as claimed in claim 1, wherein the composition comprises a preservative for preventing the growth of microorganisms.
